(19) United States
(12) Patent Application Publication
(10) Pub. No.: US 2021/0308096 A1
(43) Pub. Date: Oct. 7, 2021

(54) COMPOSITION FOR PREVENTING OR TREATING PRURITUS

(71) Applicants: INDUSTRIAL COOPERATION FOUNDATION CHONBUK NATIONAL UNIVERSITY, Jeollabuk-do (KR); STEMDR INC., Jeollabuk-do (KR)

(72) Inventors: Myung-Kwan HAN, Jeollabuk-do (KR); Hern-Ku LEE, Jeollabuk-do (KR); So-Jeong KIM, Seoul (KR); Suhn-Young IM, Gwangju (KR); Baik-Hwan CHO, Jeollabuk-do (KR); Jeong-Soo PARK, Jeollabuk-do (KR)

(73) Assignees: INDUSTRIAL COOPERATION FOUNDATION CHONBUK NATIONAL UNIVERSITY, Jeollabuk-do (KR); STEMDR INC., Jeollabuk-do (KR)

(21) Appl. No.: 17/351,399

(22) Filed: Jun. 18, 2021

Related U.S. Application Data
(63) Continuation of application No. 16/493,485, filed on Sep. 12, 2019.

(30) Foreign Application Priority Data
Mar. 14, 2017 (KR) 10-2017-0031831

(51) Int. Cl. A61K 31/353; A23L 33/10; A61P 17/04; A61K 8/49; A61Q 19/00

(52) U.S. Cl. CPC A61K 31/353; A23L 33/10; A23V 2002/00; A61K 8/498; A61Q 19/00; A61P 17/04

(57) ABSTRACT

The present disclosure relates to a composition for preventing, improving or treating pruritus by using a naturally derived ingredient. The composition of the present disclosure may be used to improve or treat pruritus caused by various causes safely and effectively without the risk of side effects.

### CROSS-REFERENCE TO RELATED APPLICATIONS

This application is a continuation of U.S. application Ser. No. 16/493,485, filed Sep. 12, 2019, which is a U.S. National Stage Entry of International Application No. PCT/KR2017/014565, filed Dec. 12, 2017, which claims priority to South Korean Application No. KR10-2017-0031831, filed Mar. 14, 2017, the entireties of which are incorporated herein by reference.

### TECHNICAL FIELD

The present disclosure relates to a composition for preventing or treating pruritus, which contains a natural ingredient.

### Background Art

Pruritus (itch) is defined as an unpleasant sensation that causes the desire to scratch the skin (Andersen H H et al., Human surrogate models of histaminergic and non-histaminergic itch, Acta Dermato-Venereologica. 95 (7): 7717. (2015)). Although it is a symptom commonly found in skin diseases and systemic diseases, its characteristics are not known sufficiently.

It is estimated that approximately 280 million people, 4% of the world population, suffer from pruritus. This is more than the 2-3% of the population suffering from psoriasis (Vos, T et al., Years lived with disability (YLDs) for 1160 sequelae of 289 diseases and injuries 19902010: a systematic analysis for the Global Burden of Disease Study 2010, Lancet. 380 (9859): 216396. (2012)).

Pruritus may be caused or aggravated by various stimuli including physical, mechanical and chemical factors. Inflammatory mediators also cause pruritus in a variety of inflammatory skin diseases. However, not all types of pruritus are associated with the mediators and pruritus caused by mechanical or electrical stimulation and dry skin may occur without regard to the mediators.

According to the National Health Information Portal (http://health.mw.go.kr), histamine, serotonin, prostaglandin E, tachykinins, cytokines, proteases, opioid peptides, platelet-activating factor, etc. are known as pruritus-causing mediators.

Histamine is synthesized by mast cells in the skin, stored in mast cell granules and secreted in response to various stimuli. Two types of histamine receptors, H1 and H2, exist in the skin. Histamine causes pruritus binding to the H1 receptor. Histamine is associated with a number of inflammatory skin diseases, including UV-induced dermatitis.

Serotonin is found in platelets. It causes pruritus by activating dermal mast cells, thereby releasing histamine, or by acting on the 5-HT3 receptor of the central nervous system.

Prostaglandin E does not cause pruritus for itself but aggravates pruritus caused by other mediators. Prostaglandin E is increased in pruritus-related skin diseases such as eczema and UV-induced inflammations.

Substance P is a member of the tachykinin neuropeptide family. It is synthesized in the dorsal root ganglion of nociceptor C fibers, transmitted to unmyelinated sensory nerve fibers and acts as a neurotransmitter. Substance P causes flare and pruritus by acting as a potent vasodilator. Substance P is released by tryptase of mast cells and causes pruritus by releasing histamine from dermal mast cells directly or via the NK-1 receptor.

Cytokines are small proteins produced in all eukaryotic cells. They act as cell-surface receptors and interleukins (IL), chemokines, interferons, etc. are included. Although IL-1 and IL-8 do not induce pruritus, human recombinant IL-2 causes severe pruritus accompanied by skin flare and eosinophilia when injected to cancer patients for the purpose of treatment.

Among proteases, kallikrein causes pruritus when injected into the skin. In addition to kallikrein, other proteases such as chymotrypsin, trypsin, papain, etc. also cause pruritus.

Opioid peptides cause pruritus by acting on the central or peripheral nervous system. There are three subtypes of opioid peptides, μ, δ and κ. It is though that the pharmacological action is exerted via the μ-receptor because it is mostly inhibited by naloxone. Morphine causes pruritus in most of patients when injected into the spinal cord and results in pruritus and flare without regard to the release of prostaglandin or histamine when injected into the dermis.

Platelet-activating factor is a potent inflammation-inducing substance secreted by a wide variety of inflammatory cells. It was reported that platelet-activating factor directly causes pruritus in animal tests. In human, it causes pruritus indirectly by inducing the secretion of histamine by mast cells.

Depending on causes, pruritus can be classified into 1) paroxysmal pruritus occurring paroxysmally, 2) skin itching including winter itch, pruritus ani, pruritus vulvae, pruritus scroti, aquagenic pruritus and scalp pruritus, 3) pruritus accompanied by internal diseases including cholestatic pruritus, chronic renal failure, malignant tumor, iron-deficiency anemia, polycythemia vera, hyperparathyroidism, hypoparathyroidism, diabetes and acquired immune deficiency syndrome, 4) pruritus accompanied by psychocutaneous disorders such as lichen simplex chronicus, prurigo, trichotillomania, neurotic excoriation, cutaneous behavior disorder, delusional parasitosis, etc. and 5) nasal pruritus associated with cold, etc., neck itch, ocular pruritus accompanied by ocular diseases such as conjunctivitis, etc., oral pruritus accompanied by dental diseases, etc.

A variety of treatment methods are currently available, including antihistamines, steroids, antibiotics, antiviral agents, antifungal agents, anesthetics, probiotics, immunosuppressants, phototherapy such as UV, etc. But, the therapeutic effect is temporary or limited depending on the type of pruritus. Use of adrenocortical hormones or corticosteroids is limited to acute or severe cases due to side effects and long-term use is undesirable. Accordingly, development of a safe therapy effective for pruritus caused by various causes is necessary.

The above description is just for helping in understanding the background of the present disclosure and should not be accepted as being acknowledged as widely known by those of ordinary skill in the related art.

### DISCLOSURE

### Technical Problem

The inventors of the present disclosure have made efforts to find naturally derived substances which can be prescribed safely for pruritus caused by various causes without the risk of side effects. As a result, they have identified that some isoflavone-based compounds are very effective for pruritus caused by various causes and have completed the present disclosure.

The present disclosure is directed to providing a pharmaceutical composition for preventing or treating pruritus.

The present disclosure is also directed to providing a food composition for preventing or improving pruritus.

The present disclosure is also directed to providing a cosmetic composition for preventing or improving pruritus.

Other features and aspects will be apparent from the following detailed description, the drawings, and the claims.

### Technical Solution

In an aspect, the present disclosure provides a pharmaceutical composition for preventing or treating pruritus, containing a compound of Chemical Formula 1 as an active ingredient:

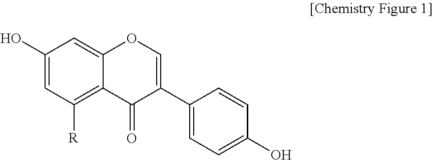

wherein R is H or OH.

As a result of making efforts to find naturally derived substances which can be prescribed safely for pruritus caused by various causes, the inventors of the present disclosure have identified that the compound of Chemical Formula 1 is very effective for pruritus caused by various causes.

According to the experiments conducted by the inventors of the present disclosure, the compound of the present disclosure shows significant therapeutic effect for pruritus caused by various causes, including histamine-induced pruritus, chloroquine-induced pruritus, DNCB (2,4-dinitrochlorobenzene)-induced pruritus, SLIGRL (H-Ser-Leu-Ile-Gly-Arg-Leu-NH₂)-induced pruritus, TSLP (thymic stromal lymphopoietin)-induced pruritus, etc. In addition, the improving and therapeutic effect for pruritus are observed only in genistein and daidzein, not in all isoflavone-based compounds (genistein, daidzein, glycitein and equol) (Example 6).

Genistein, one of the active ingredients of the present disclosure, has a structure of Chemical Formula 2. With excellent antioxidant effect (Han, Rui-Min et al., Comparison of Flavonoids and Isoflavonoids as Antioxidants, Journal of Agricultural and Food Chemistry. 57 (9): 37805. (2009)), it is commonly used as a cosmetic additive. However, nothing is known about its effect in improvement of pruritus.

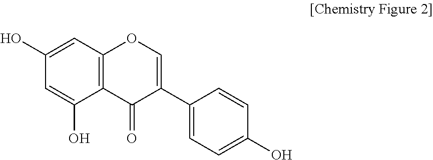 [SMILES: O=c1c(-c2ccc(O)cc2)coc2cc(O)cc(O)c12]

Daidzein, another active ingredient of the present disclosure, has a structure of Chemical Formula 3. It is known to activate PPARs (Dang Z. C. et al., The Balance between Concurrent Activation of ERs and PPARs Determines Daidzein-Induced Osteogenesis and Adipogenesis, Journal of Bone and Mineral Research. 19 (5): 853861. (2004)) and to lower the risk of breast cancer when administered with high concentrations (de Lemos, M. L., Effects of soy phytoestrogens genistein and daidzein on breast cancer growth, Annals of Pharmacotherapy. 35 (9): 11118-11121 (2001)). However, nothing is known about the daidzein's effect in improvement of pruritus.

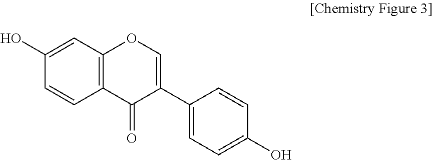 [SMILES: O=c1c(-c2ccc(O)cc2)coc2cc(O)ccc12]

In the present disclosure, the term “pruritus” or “itch” is not specially limited but is interpreted to include paroxysmal pruritus, winter itch, pruritus ani, pruritus vulvae, pruritus scroti, aquagenic pruritus, scalp pruritus, nasal pruritus, neck itch, oral pruritus, ocular pruritus, pruritus accompanied by internal diseases such as cholestatic pruritus, chronic renal failure, malignant tumors, iron-deficiency anemia, polycythemia vera, hyperparathyroidism, hypoparathyroidism, diabetes, acquired immune deficiency syndrome, etc. and pruritus accompanied by psychocutaneous disorders such as lichen simplex chronicus, prurigo, trichotillomania, neurotic excoriation, cutaneous behavior disorder, delusional parasitosis, etc.

Paroxysmal pruritus is paroxysmally occurring pruritus and is found in lichen simplex chronicus, dermatitis, etc.

Winter itch occurs in about 50% of people who are 70 years or older and should be distinguished from pruritus caused by pruritic skin diseases such as scabies, lichen planus, etc. or systemic diseases. In women, it may occur as a symptom of the postmenopausal syndrome. Decreased water content and gradually declining sebum secretion of the aged skin are the major cause of skin dryness and fine cracks and scales are frequently found in upper limbs and tibias.

Pruritus ani is the unpleasant irritation around the anus, causing the desire to scratch, and psychogenic factors are often involved. It may occur regardless of age but occurs more frequently in elderly people. However, not all pruritus ani is psychogenic and contamination and irritation stimulation around the anus may be the cause. The irritation may be aggravated by colorectal diseases such as fissure, hemorrhoid, fistula and chronic diarrhea, spicy food, medicine, etc. Various infectious diseases such as staphylococcus, streptococcus, fungi, candida, herpes simplex virus, etc. may cause this pruritus. Among them, candida infection is the most common cause and cracking and swelling of the skin are observed. Skin diseases such as psoriasis, seborrhoic dermatitis, lichen planus, etc. around the anus may also cause severe pruritus and lesions can be found in other areas, too. Anal neurodermatitis, characterized by violent itching leading to repeated rubbing until bleeding, may show symptoms similar to those of lichen simplex chronicus in other areas.

The most common cause of pruritus vulvae is candida infection. Other causes include trichomonal vaginitis and contact dermatitis by pads, contraceptives, vaginal washes, condoms, etc. In the elderly, lichen sclerosus is a common cause. Severe pruritus may occur also in Fox-Fordyce disease. However, temporary pruritus vulvae may also be caused by rubbing, sweating or vulvar congestion during pregnancy.

Regarding pruritus scroti, the scrotum of an adult is immune to fungal infection like the scalp of an adult but is the area where lichen simplex chronicus occurs frequently. It is mainly caused by psychogenic factors. Lichenification occurs severely and the symptom often lasts several years despite intensive treatment.

Aquagenic pruritus is characterized by severe, unpleasant, prickling-like itching, which occurs within several minutes after contact with water, and lasts for about 1 hour. It is irrelevant to the temperature of the water and no appreciable change is observed in the skin. For some patients, it may be caused by the change in ambient temperature. About ⅓ of the patients have family history. The symptoms are usually chronic and unresponsive to treatment. Although increased histamine level is observed in the skin and blood, histamine is not considered the only mediator because the symptom is not alleviated by antihistamines. Differentiation from polycythemia vera is necessary because the symptoms are similar.

Scalp pruritus may occur as an independent symptom without appreciable lesions on the scalp. It is found in middle-aged or elderly people, but the cause is not known. The itchiness is very severe and occurs paroxysmally. It is further aggravated by fatigue or stress. It needs to be distinguished from dermatitis herpetiformis, lichen simplex chronicus, seborrhoic dermatitis, psoriasis, etc.

Biliary cirrhosis is accompanied by severe systemic pruritus. The pruritus is associated with the increased level of bile acid in blood plasma. Severe pruritus may be induced by direct application of bile acid of a concentration that can cause pruritus clinically onto the blistery skin lesion.

Pruritus occurs in about 20-50% of chronic renal failure patients receiving hemodialysis therapy. The pruritus occurs topically or systemically. In most cases, the symptom is aggravated during hemodialysis. But, the symptom may be alleviated temporarily. It is reported that there is no direct relationship between the blood levels of histamine, urea and creatinine and the degree of pruritus. Some patients have xeroderma but most have normal skin and use of moisturizers do not alleviate or reduce the symptom.

Regarding malignant tumors, extensive diagnosis of malignant tumors is necessary if systemic pruritus occurs in middle-aged or elderly people without clear causes. Pruritus occurs consistently in 15-25% of patients with Hodgkin's disease (a representative disease causing the swelling of lymph nodes). It is often accompanied by burning pain and flushing and the cause is it known. Systemic pruritus may also occur in leukemia.

Iron deficiency may also be a cause of pruritus. Decreased pruritus was reported in patients with polycythemia vera and iron deficiency when iron supplement was orally administered.

About 50% of patients with polycythemia vera experience severe pruritus within several minutes after contact with water and the system continues for about 15-60 minutes. It is called bath itch because it usually occurs after taking a bath. There is no appreciable change in the skin and the symptom occurs regardless of the water temperature. However, increased histamine level is found in serum and urine. Platelet aggregation is considered to lead to the release of several pruritus mediators including histamine.

Severe systemic pruritus may occur in hyperparathyroidism. Increased skin temperature due to increased blood flow and decreased itch threshold resulting therefrom are the cause of the systemic pruritus. Hypoparathyroidism- and myxedema-associated systemic pruritus may be related to the severe dryness of the skin. In both diseases, pruritus may occur around the genitals due to mucocutaneous candidiasis.

In some diabetic patients, pruritus may occur around the anus and genitals due to mucocutaneous candidiasis. However, systemic pruritus may also occur in some patients.

One of the important symptoms of acquired immune deficiency syndrome is pruritus. The cause of pruritus occurring in patients with acquired immune deficiency syndrome includes scabies, pediculosis, candidiasis, seborrhoic dermatitis, renal failure, cholestasis, etc. Often, systemic papule or pigmented rash causing characteristically severe pruritus occurs.

Lichen simplex chronicus is a disease characterized by repeated rubbing or scratching of the skin, causing thick, leathery skin. Lichen simplex chronicus may occur secondarily in normal skin as a result of repeated pruritus. It is more common in 30s to 50s and is seen more often in women compared to men.

Prurigo is a disease characterized by multiple nodules and severe pruritus. It does not cured well and tends to last for a long time. Its cause is not known well and anemia, liver disease, HIV, pregnancy, renal failure, mental stress, etc. may be the cause.

Trichotillomania is an impulse control disorder characterized by an abnormal urge that results in the pulling out of one's hair. Mental and social stresses are the cause. Stress in family or school, sibling rivalry, move, hospitalization of parents, mother-daughter relationship, etc. may be the cause. It occurs in nearly all age groups from children to adults.

Neurotic excoriation is a disease characterized by the repeated urge to pick and gouge one's own skin, often leading to skin lesions. Patients admit that their behavior is responsible for the lesions but cannot resist it. It can occur at any age but occurs more frequently in middle-aged women. It often occurs due to mental stress. It often occurs on skin lesion areas such as pruritus, insect sting, etc. Neurotic excoriation also correlates with depression, obsessive-compulsive disorder and anxiety. This symptom occurs more frequently in people with obsessive, stubborn, supervisory characteristics and perfectionism tendencies with fear for failure.

Factitious dermatitis is a dermatitis occurring from intentional self-inflicted skin damage in an attempt to attract sympathy or avoid responsibility. Skin lesions are produced mechanically or by chemicals, corrosives, etc. In addition, nails, sharp instruments, hot metals, etc. are used too. The patient harms himself/herself in order to satisfy psychological needs. It occurs more frequently in women and can occur in all age groups. Most patients are childish and dependent and have impulse control disorders.

Cutaneous behavior disorder is an obsessive self-inflicting behavior repeated for a long time. The self-inflicting behavior is often a suicidal attempt and, in adolescence, it may be attempted to show off bravery.

Delusional parasitosis is a delusional disorder in which patients incorrectly believe they are infested with parasite. It is a chronic, monosymptomatic hypochondriasis without damage to personality or thinking ability. Patients provide pieces of skin, lint, tissue paper, tape, etc. and ask for parasite examination. It is reported that 2-3% of the patients have experienced parasitic infection before.

Others may include nasal pruritus accompanied by nasal diseases such as rhinitis, ocular pruritus accompanied by ocular diseases such as conjunctivitis and oral pruritus accompanied by dental diseases.

The active ingredient used in the composition of the present disclosure includes, not only the compound of Chemical Formula 1 itself, but also its pharmaceutically acceptable salt, hydrate, solvate or prodrug.

The term “pharmaceutically acceptable salt” refers to a salt of the compound of Chemical Formula 1 which exerts the desired pharmacological effect, i.e., the activity of improving pruritus. This salt may be formed by using an inorganic acid (e.g., hydrochloride, hydrobromide and hydroiodide) or an organic acid (e.g., acetate, adipate, alginate, aspartate, benzoate, benzenesulfonate, p-toluenesulfonate, bisulfate, sulfamate, sulfate, sulfamate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, 2-hydroxyethanesulfate, lactate, maleate, methanesulfonate, 2-naphthalenesulfonate, nicotinate, oxalate, tosylate and undecanoate).

The term “pharmaceutically acceptable hydrate” refers to a hydrate of the compound of Chemical Formula 1 which exerts the desired pharmacological effect. The term “pharmaceutically acceptable solvate” refers to a solvate of the compound of Chemical Formula 1 which exerts the desired pharmacological effect. The hydrate and the solvate may also be prepared by using the above-described acids.

The term “pharmaceutically acceptable prodrug” refers to a derivative of the compound of Chemical Formula 1 which has to undergo bioconversion prior to exhibiting the pharmacological effect of the compound of Chemical Formula 1. The prodrug is prepared to improve chemical stability, patient compliance, bioavailability or organ selectivity, improve convenience of preparation, prolong the duration of action or reduce side effects. The prodrug of the present disclosure may be prepared easily from the compound of Chemical Formula 1 according to a method commonly employed in the art (e.g., Burger's Medicinal Chemistry and Drug Chemistry, 5th ed., 1: 172-178 and 949-982 (1995)).

A pharmaceutically acceptable carrier contained in the pharmaceutical composition of the present disclosure may be one commonly used in the art and may include lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc., although not being limited thereto. The pharmaceutical composition of the present disclosure may further contain, in addition to these ingredients, a lubricant, a wetting agent, a sweetener, a flavor, an emulsifier, a suspending agent, a preservative, etc. Suitable pharmaceutically acceptable carriers and formulations are described in detail in Remington's Pharmaceutical Sciences (19th ed., 1995).

The pharmaceutical composition of the present disclosure may be administered orally or parenterally. The parenteral administration can be made nasally, ocularly, intravenously, subcutaneously, intramuscularly, intraperitoneally, transdermally, etc.

An adequate administration dosage of the pharmaceutical composition of the present disclosure may vary depending on such factors as formulation method, administration method, patient's age, body weight and sex, pathological condition, diet, administration time, administration route, excretion rate and responsiveness. An ordinarily skilled physician can easily determine and prescribe an administration dosage which is effective for the desired treatment or prevention. In a specific exemplary embodiment of the present disclosure, a daily administration dosage of the pharmaceutical composition of the present disclosure is 0.001-100 mg/kg.

The pharmaceutical composition of the present disclosure may be formulated as a unit dosage form or in a multiple-dosage receptacle by using a pharmaceutically acceptable carrier and/or excipient according to a method that can be easily carried out by those of ordinary skill in the art to which the present disclosure belongs. The formulation may be in the form of a solution in an oily or aqueous medium, a suspension, an emulsion, an extract, a powder, a granule, a tablet or a capsule and may further contain a dispersant or a stabilizer.

The pharmaceutical composition of the present disclosure may also be prepared into a formulation for external application to the skin, an aerosol, a spray, an eye drop, an oral medication or an injection.

In another aspect, the present disclosure provides a food composition for preventing or improving pruritus, which contains a compound of Chemical Formula 1 as an active ingredient:

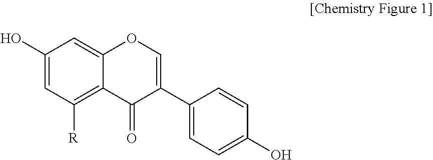

wherein R is H or OH.

When the composition of the present disclosure is prepared as a food composition, it may further contain, in addition to the compound of Chemical Formula 1 as the active ingredient, an ingredient commonly added during preparation of food, for example, a protein, a carbohydrate, a fat, a nutrient, a seasoning agent or a flavoring agent. Examples of the carbohydrate are common sugars such as monosaccharides, e.g., glucose, fructose, etc., disaccharides, e.g., maltose, sucrose, oligosaccharides, etc., and polysaccharides, e.g., dextrin, cyclodextrin, etc., and sugar alcohols such as xylitol, sorbitol, erythritol, etc. As the flavoring agent, a natural flavor (thaumatin, stevia extract (e.g., rebaudioside A, glycyrrhizin, etc.), etc.) or a synthetic flavor (saccharin, aspartame, etc.) may be used.

For example, when the composition of the present disclosure is prepared as a drink, it may further contain, in addition to the compound of Chemical Formula 1, citric acid, fructose syrup, sucrose, glucose, acetic acid, malic acid, fruit juice, eucommia bark extract, jujube extract, licorice extract, etc.

The composition of the present disclosure exerts very superior action in improving pruritus. In addition, the composition of the present disclosure is very safe for the human body because it uses an isoflavone-based compound with proven biological safety as an active ingredient.

In another aspect, the present disclosure provides a cosmetic composition for preventing or improving pruritus, which contains a compound of Chemical Formula 1 as an active ingredient:

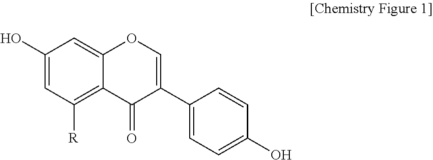

wherein R is H or OH.

When the composition of the present disclosure is prepared as a cosmetic composition, the composition of the present disclosure may further contain, in addition to the compound of Chemical Formula 1, an ingredient commonly used in a cosmetic composition, for example, a commonly used adjuvant such as an antioxidant, a stabilizer, a solubilizer, a vitamin, a pigment and a flavor and a carrier. Besides, the composition of the present disclosure may further contain, in addition to the compound of Chemical Formula 1, a pruritus-improving agent which has been used hitherto within a range not negatively affecting the (pruritus-improving) action of the active ingredient.

As the carrier, purified water, a monohydric alcohol (ethanol or propyl alcohol), a polyhydric alcohol (glycerol, 1,3-butylene glycol or propylene glycol), a higher fatty acid (palmitic acid or linoleic acid), an oil or fat (wheat germ oil, camellia oil, jojoba oil, olive oil, squalene, sunflower oil, macadamia nut oil, avocado oil, hydrogenated soybean lecithin or fatty acid glyceride), etc. may be used, although not being limited thereto. If necessary, a surfactant, a sterilizer, an antioxidant, a UV absorber, an anti-inflammatory agent or a cooling agent may be further added.

The surfactant may be selected from a group consisting of polyoxyethylene, hydrogenated castor oil, polyoxyethylene oleyl ether, polyoxyethylene monooleate, polyoxyethylene glyceryl monostearate, sorbitan monostearate, sorbitan, sucrose fatty acid ester, hexaglyceryl monolaurate, polyoxyethylene-reduced lanolin, POE, glyceryl pyroglutamate, isostearic acid, diester, N-acetylglutamine and isostearyl ester.

The sterilizer may be selected from a group consisting of hinokitiol, triclosan, chlorhexidine gluconate, phenoxyethanol, resorcin, isopropylmethylphenol, azulene, salicylic acid and zinc pyrithione.

As the antioxidant, any of butylated hydroxyanisole, gallic acid, propyl gallate and erythorbic acid may be used.

As the UV absorber, any of a benzophenone such as dihydroxybenzophenone, etc., melanin, ethyl p-aminobenzoate, p-dimethylaminobenzoic acid 2-ethylhexyl ester, cinoxate, p-methoxycinnamonic acid 2-ethylhexyl ester, 2-(2-hydroxy-5-methylphenyl)benzotriazole, urocanic acid and fine metal oxide particle may be used.

As the anti-inflammatory agent, dipotassium glycyrrhetinate or allantoin may be used. And, as the cooling agent, capsicum tincture or 1-menthol may be used.

The composition may be prepared into any formulation in which the compound of Chemical Formula 1 can be mixed as an active ingredient. Examples of the cosmetic formulation for improving pruritus include a tonic, a shampoo, a rinse, a hair conditioner, a hair spray, a powder, a gel, a cream, an essence, a lotion, a sol-gel, an emulsion, an oil, a wax, a spray, a mist, etc., although not being limited thereto. In addition, it may be prepared as a mask pack containing the compound of Chemical Formula 1.

### Advantageous Effects

The features and advantages of the present disclosure may be summarized as follows: (i) The present disclosure provides a composition for preventing, improving or treating pruritus by using a naturally derived ingredient.

(ii) The composition of the present disclosure may be used to improve or treat pruritus caused by various causes safely and effectively without the risk of side effects.

### DESCRIPTION OF DRAWINGS

FIG. 1 compares the pruritus-alleviating effect of genistein, daidzein, glycitein and equol at concentrations of 1 μM and 10 μM after inducing pruritus by treating with histamine (p<0.01 versus histamine-treated group).

FIG. 2 compares the pruritus-alleviating effect of genistein, daidzein, glycitein and equol at concentrations of 1 μM and 10 M after inducing pruritus by treating with chloroquine (p<0.01 versus chloroquine-treated group).

FIG. 3 compares the pruritus-alleviating effect of genistein, daidzein, glycitein and equol at concentrations of 1 μM and 10 M after inducing pruritus by treating with DNCB (p<0.01 versus DNCB-treated group).

FIG. 4 compares the pruritus-alleviating effect of genistein, daidzein, glycitein and equol at concentrations of 1 μM and 10 M after inducing pruritus by treating with SLIGRL (p<0.05 versus SLIGRL-treated group).

FIG. 5 compares the pruritus-alleviating effect of genistein, daidzein, glycitein and equol at concentrations of 1 μM and 10 M after inducing pruritus by treating with TSLP (p<0.05 versus TSLP-treated group).

### BEST MODE

Hereinafter, the present disclosure will be described in detail through examples. However, the following examples are for illustrative purposes only and it will be apparent to those of ordinary skill in the art that the scope of the present disclosure is not limited by the examples.

### EXAMPLES

### <Example 1> Test Animals

Six-week-old, specific-pathogen-free male BALB/c mice were purchased from Orient Bio Korea Inc. (Gapyeong, Gyeonggi-do, Korea) and housed in a laminar flow cabinet. They were provided with standard solid chows ad libitum. At the onset of the test, the mice were 7-8 weeks old. All animal care and use were performed in accordance with the protocol of the Institutional Animal Care and Use Committee of the Chonbuk National University Medical School.

### <Example 2> Preparation of Reagents

Isoflavone-based compounds genistein, daidzein, glycitein and equol were purchased from Sigma. They were dissolved in DMSO and diluted with distilled water. Care was taken such that the content of DMSO did not exceed 0.1%. Histamine, chloroquine and DNCB were also purchased from Sigma.

### <Example 3> Induction of Pruritus

1. Histamine- and Chloroquine-Induced Pruritus

Pruritus was induced by subcutaneously injecting histamine and chloroquine to the mice. Specifically, 0.1 mL of 2 mg/mL histamine and 4 mg/mL chloroquine were injected respectively.

2. DNCB (2,4-Dinitrochlorobenzene)-Induced Pruritus

Pruritus was induced with DNCB. Specifically, after removing hair from the right ear of the mouse, 20 μL of 1% DNCB dissolved in a mixture of olive oil and ethanol (1:4) was applied for sensitization. 10 μL of 0.5% DNCB was applied on the same ear 7 days later (Exp. Dermatol., 2002, 11:285-291).

*1073. SLIGRL (H-Ser-Leu-Ile-Gly-Arg-Leu-NH₂)-Induced Pruritus

Pruritus was induced with SLIGRL. Specifically, 20 μg of SLIGRL was dissolved in 0.1 mL of physiological saline and injected subcutaneously to induce pruritus.

4. TSLP (Thymic Stromal Lymphopoietin)-Induced Pruritus

Pruritus was induced with TSLP. 0.67 μg of TSLP was dissolved in 0.1 mL of physiological saline and injected subcutaneously to induce pruritus.

### <Example 4> Measurement of Pruritus

Pruritus was evaluated using MicroAct (Neuroscience, Inc., Tokyo, Japan) according to the method established by the company. The mouse was anesthetized and a magnet piece was inserted into the foot skin of the left hind leg. The mouse was put in a chamber surrounded by a coil and the number of scratching actions was recorded by a computer based on the electrical field generated by the scratching. The experimental conditions of the MicroAct were set as follows. Threshold p-p limit, 0.125 V; threshold minimum duration, 0.2 s; maximum amplitude range, 0.5 V; minimum amplitude range, 0.05 V; maximum frequency, 20 Hz; minimum frequency, 5 Hz. Recording was made only for 3 or more consecutive scratching actions.

### <Example 5> Statistical Analysis

All the experiments were performed for at least 3 times and 2-3 mice were used per each group. Statistical data were represented by means and standard deviations. Statistical comparison was conducted by one-way ANOVA and the Fisher test. Significant difference between the groups was determined by the unpaired Student's t-test. The significant level of the p-value was less than 0.05.

### <Example 6> Effect of Inhibiting Pruritus

1. Effect of Inhibiting Histamine-Induced Pruritus

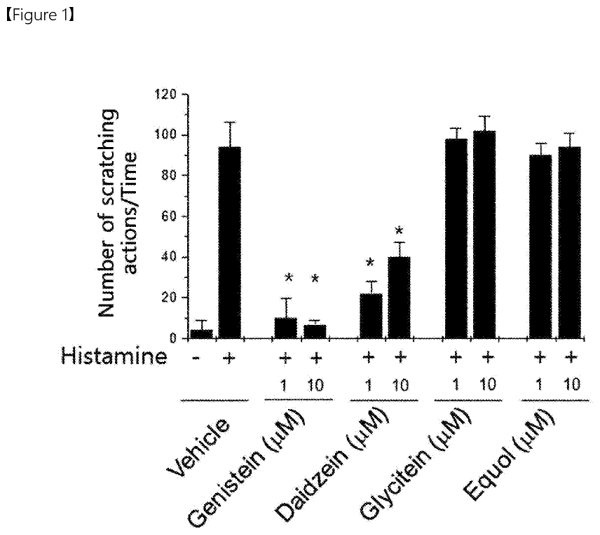

In order to measure the effect of inhibiting histamine-induced pruritus of genistein, daidzein, glycitein and equol, 100 μg of histamine was dissolved in 0.1 mL of physiological saline and injected subcutaneously to induce pruritus. After injecting each of genistein, daidzein, glycitein and equol at 1 μM and 10 μM together with the histamine, the number of scratching actions was counted for 1 hour. Genistein and daidzein significantly inhibited histamine-induced pruritus at concentrations of 1 μM and 10 μM (p<0.01) (FIG. 1). However, glycitein and equol did not inhibit histamine-induced pruritus at the concentrations (FIG. 1).

2. Effect of Inhibiting Chloroquine-Induced Pruritus

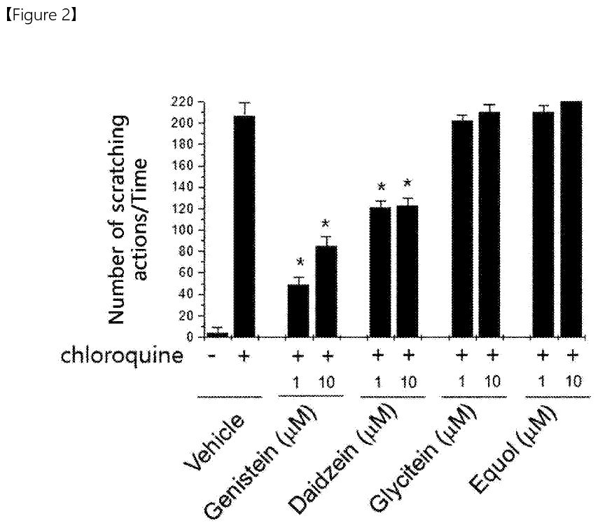

In order to measure the effect of inhibiting chloroquine-induced pruritus of genistein, daidzein, glycitein and equol, 200 μg of chloroquine was dissolved in 0.1 mL of physiological saline and injected subcutaneously to induce pruritus. After injecting each of genistein, daidzein, glycitein and equol at 1 μM and 10 μM together with the chloroquine, the number of scratching actions was counted for 1 hour. Genistein and daidzein significantly inhibited chloroquine-induced pruritus at concentrations of 1 μM and 10 μM (p<0.01) (FIG. 2). However, glycitein and equol did not inhibit chloroquine-induced pruritus at the concentrations (FIG. 2).

3. Effect of Inhibiting DNCB-Induced Pruritus

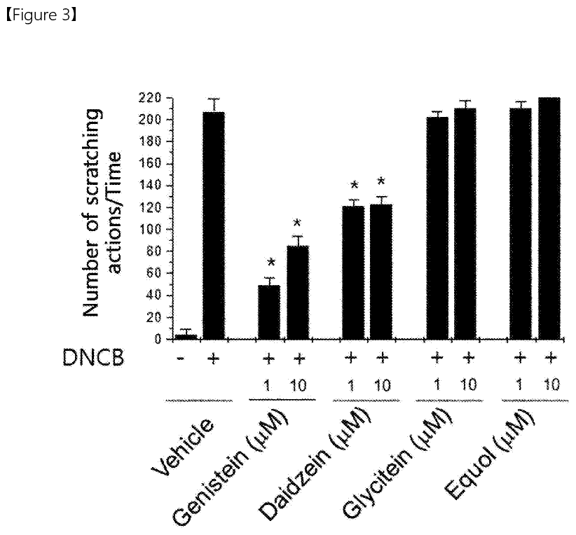

In order to measure the effect of inhibiting DNCB-induced pruritus of genistein, daidzein, glycitein and equol, 20 μL of a 1% DNCB solution was applied on the right ear of the mouse for 7 days for sensitization. 10 μL of 0.5% DNCB was applied on the same ear 7 days later, 1 μM or 10 μM genistein, daidzein, glycitein or equol and 1% DNCB dissolved in physiological saline was applied on the right ear (physiological saline was applied for a negative control group) and the number of scratching actions was counted for 1 hour. Genistein and daidzein significantly inhibited DNCB-induced pruritus at concentrations of 1 μM and 10 μM (p<0.01) (FIG. 3). However, glycitein and equol did not inhibit DNCB-induced pruritus at the concentrations (FIG. 3).

4. Effect of Inhibiting SLIGRL-Induced Pruritus

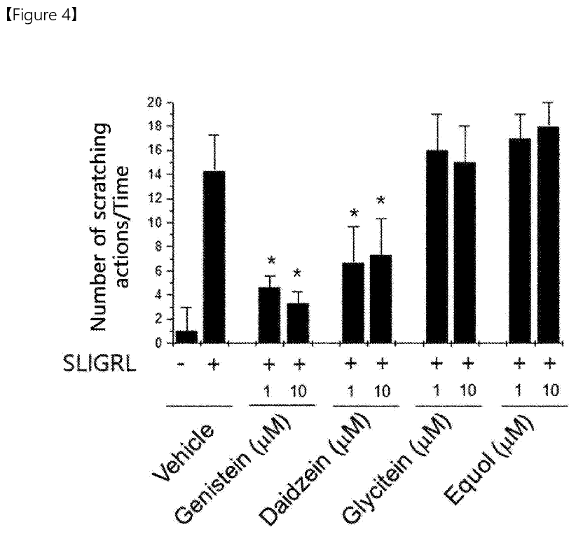

In order to measure the effect of inhibiting pruritus induced by SLIGRL, which is an agonist for the protease activated receptor 2, of genistein, daidzein, glycitein and equol, 20 μg of SLIGRL was dissolved in 0.1 mL of physiological saline and injected subcutaneously to induce pruritus. After injecting each of genistein, daidzein, glycitein and equol at 1 μM and 10 μM together with the SLIGRL, the number of scratching actions was counted for 1 hour. Genistein and daidzein significantly inhibited SLIGRL-induced pruritus at concentrations of 1 μM and 10 μM (p<0.05) (FIG. 4). However, glycitein and equol did not inhibit SLIGRL-induced pruritus at the concentrations (FIG. 4).

5. Effect of Inhibiting TSLP-Induced Pruritus

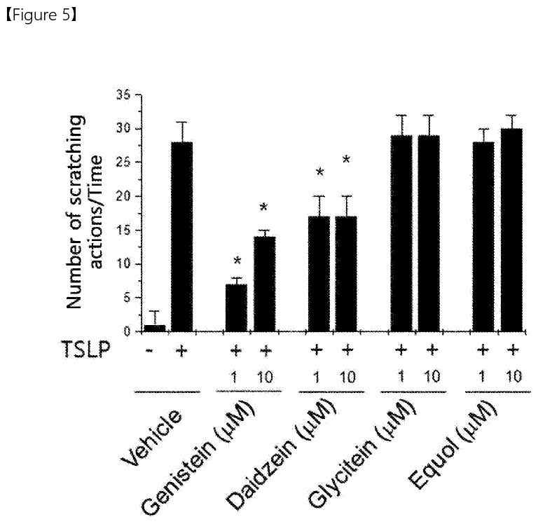

In order to measure the effect of inhibiting thymic stromal lymphopoietin (TSLP)-induced pruritus of genistein, daidzein, glycitein and equol, 0.67 μg of TSLP solution dissolved in 0.1 mL of physiological saline and injected subcutaneously to induce pruritus. After injecting each of genistein, daidzein, glycitein and equol at 1 μM and 10 μM together with the TSLP, the number of scratching actions was counted for 1 hour. Genistein and daidzein significantly inhibited TSLP-induced pruritus at concentrations of 1 μM and 10 μM (p<0.05) (FIG. 5). However, glycitein and equol did not inhibit TSLP-induced pruritus at the concentrations (FIG. 5).

While the present disclosure has been described with respect to the specific embodiments, it will be apparent to those skilled in the art that various changes and modifications may be made without departing from the spirit and scope of the disclosure as defined in the following claims and their equivalents.

1. A method of preventing or treating pruritus, comprising:
providing a pharmaceutical composition to an individual in need thereof, wherein the pharmaceutical composition comprises a compound of Chemical Formula 1 as an active ingredient: 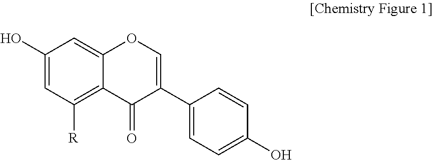
wherein R is H or OH.

2. The method according to claim 1, wherein the pruritus is selected from a group consisting of paroxysmal pruritus, winter itch, pruritus ani, pruritus vulvae, pruritus scroti, aquagenic pruritus, scalp pruritus, nasal pruritus, neck itch, oral pruritus and ocular pruritus.

3. The method according to claim 1, wherein the pruritus is pruritus accompanied by an internal disease selected from a group consisting of cholestatic pruritus, chronic renal failure, malignant tumor, iron-deficiency anemia, polycythemia vera, hyperparathyroidism, hypoparathyroidism, diabetes and acquired immune deficiency syndrome.

4. The method according to claim 1, wherein the pruritus is pruritus accompanied by a psychocutaneous disorder selected from a group consisting of lichen simplex chronicus, prurigo, trichotillomania, neurotic excoriation, cutaneous behavior disorder and delusional parasitosis.

5. The method according to claim 1, wherein the composition is in a form selected from a group consisting of a formulation for external application to the skin, an aerosol, a spray, an eye drop, an oral medication and an injection.

6. The method according to claim 1, wherein the composition is formulated in the form selected from the group consisting of a solution in an oily or aqueous medium, a suspension, an emulsion, an extract, a powder, a granule, a tablet, and a capsule.

7. The method according to claim 1, wherein the pharmaceutical composition is administered orally.

8. The method according to claim 1, wherein the pharmaceutical composition is administered parenterally from a group selected from the group consisting of nasally, ocularly, intravenously, subcutaneously, intramuscularly, intraperitoneally, and transdermally.

9. The method according to claim 1, wherein the pharmaceutical composition is administered as a daily dosage in the amount of 0.001-100 mg/kg.

10. The method according to claim 1, wherein R is OH.

11. The method according to claim 1, wherein R is H.

12. A method for preventing or improving pruritus, comprising:
providing a food composition comprising a compound of Chemical Formula 1 as an active ingredient to be ingested by an individual in need thereof: 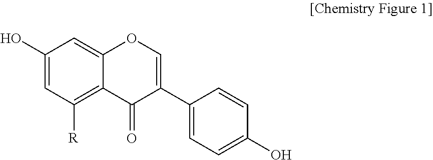
wherein R is H or OH.

13. The method according to claim 12, wherein the food composition further comprises a at least one ingredient selected from the group consisting of a protein, a carbohydrate, a fat, a nutrients, a seasoning agent, and a flavoring agent.

14. The method according to claim 12, wherein the food composition is a drink.

15. A method for preventing or improving pruritus, comprising:
providing a cosmetic composition comprising a compound of Chemical Formula 1 as an active ingredient; and
applying the cosmetic composition to the skin of an individual in need thereof: 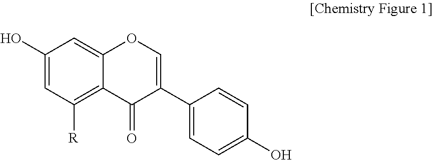
wherein R is H or OH.

16. The method of claim 15, wherein the cosmetic composition is in the form of a tonic, a shampoo, a rinse, a hair conditioner, a hair spray, a powder, a gel, a cream, an essence, a lotion, a sol-gel, an emulsion, an oil, a wax, a spray or a mist.

17. The method of claim 15, wherein the cosmetic composition further comprises an ingredient selected from the group consisting of an antioxidant, a stabilizer, a solubilizer, a vitamin, a pigment, a flavor, and a carrier.

18. The method of claim 15, wherein the cosmetic composition is incorporated into a mask that is worn by the individual.

19. The method according to claim 15, wherein R is OH.

20. The method according to claim 15, wherein R is H.
